(19) United States
(12) Patent Application Publication
(10) Pub. No.: US 2024/0016375 A1
(43) Pub. Date: Jan. 18, 2024

(54) Medical Device Reprocessor With Multi-Source Dispenser

(71) Applicant: ASP Global Manufacturing GMBH, Schaffhausen (CH)

(72) Inventor: Sungwook Yang, Irvine, CA (US)

(73) Assignee: ASP Global Manufacturing GMBH, Schaffhausen (CH)

(21) Appl. No.: 18/036,056

(22) PCT Filed: Nov. 3, 2021
(86) PCT No.: PCT/IB2021/000784

§ 371 Date: May 9, 2023

Related U.S. Application Data
(60) Provisional application No. 63/112,727, filed on Nov. 12, 2020.

(51) Int. Cl. A61B 1/12

(52) U.S. Cl. CPC A61B 1/125; A61B 1/123

(57) ABSTRACT

A system and method reprocesses an internal channel of a medical device. The reprocessing system includes one or more valves, two or more fluid sources with each associated with a respective one of the valves, a pump that is able to dispense fluid from each of the two or more fluid sources, and a measuring device that measures a parameter of the fluid flow, where the parameter may be a fluid flow volume or a fluid flow time. The method includes using the pump and measuring device to dispense two or more fluids in sequence such that the pump and measuring device is shared among the two or more fluids.

### PRIORITY

The application claims priority to U.S. Provisional Patent Application 63/112,727, entitled “Medical Device Reprocessor with Multi-Source Dispenser,” filed Nov. 12, 2020, the disclosure of which is incorporated by reference herein.

### BACKGROUND

The below discussion relates to the reprocessing (i.e., decontamination) of endoscopes and other instruments that are used in medical procedures. In particular, the below discussion relates to an apparatus and a method that may be used to reprocess a medical device such as an endoscope after the medical device has been used in a first medical procedure, such that the medical device may be safely used in a subsequent medical procedure. While the below discussion will speak mainly in terms of an endoscope, it should be understood that the discussion may also equally apply to certain other medical devices.

An endoscope may have one or more working channels or lumens extending along at least a portion of the length of the endoscope. Such channels may be configured to provide a pathway for passage of other medical devices, etc., into an anatomical region within a patient. These channels may be difficult to clean and/or disinfect using certain primitive cleaning and/or disinfecting techniques. Thus, the endoscope may be placed in a reprocessing system that is particularly configured to clean endoscopes, including the channels within endoscopes. Such an endoscope reprocessing system may wash and disinfect the endoscope. Such an endoscope reprocessing system may include a basin that is configured to receive the endoscope, with a pump that flows cleaning fluids over the exterior of the endoscope within the basin. The system may also include ports that couple with the working channels of the endoscope and associated pumps that flow cleaning fluids through the working channels of the endoscope. The process executed by such a dedicated endoscope reprocessing system may include a detergent washing cycle, followed by a rinsing cycle, followed by a sterilization or disinfection cycle, followed by another rinsing cycle. The sterilization or disinfection cycle may employ disinfectant solution and water rinses. The final rinsing cycle concludes with purging the endoscope channels with compressed air. Optionally, the process may further include an alcohol rinsing cycle in which the endoscope channels are filled with alcohol and then purged with compressed air to facilitate drying of the channels and thereby enhancing the decontamination effects of the process.

Examples of systems and methods that may be used to reprocess a used endoscope are described in U.S. Pat. No. 6,986,736, entitled “Automated Endoscope Reprocessor Connection with Integrity Testing,” issued Jan. 17, 2006, the disclosure of which is incorporated by reference herein; U.S. Pat. No. 7,479,257, entitled “Automated Endoscope Reprocessor Solution Testing,” issued Jan. 20, 2009, the disclosure of which is incorporated by reference herein; U.S. Pat. No. 7,686,761, entitled “Method of Detecting Proper Connection of an Endoscope to an Endoscope Reprocessor,” issued Mar. 30, 2010, the disclosure of which is incorporated by reference herein; U.S. Pat. No. 8,246,909, entitled “Automated Endoscope Reprocessor Germicide Concentration Monitoring System and Method,” issued Aug. 21, 2012, the disclosure of which is incorporated by reference herein; U.S. Pat. Pub. No. 20190076009A1, entitled “Apparatus and Method to Asynchronously Fill and Purge Channels of Endoscope Simultaneously,” published Mar. 14, 2019, the disclosure of which is incorporated by reference herein; and U.S. Pat. Pub. No. 20190076567A1, entitled “Apparatus and Method to Repeatedly Fill and Purge Channels of Endoscope,” published Mar. 14, 2019, the disclosure of which is incorporated by reference herein. An example of a commercially available endoscope reprocessing system is the EVOTECH® Endoscope Cleaner and Reprocessor (ECR) by Advanced Sterilization Products of Irvine, California.

While a variety of systems and methods have been made and used to reprocess medical devices, it is believed that no one prior to the inventor(s) has made or used the technology as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a cross-sectional side view of proximal and distal portions of an endoscope that may be decontaminated using a reprocessing system;

FIG. 2 depicts a front elevational view of an exemplary reprocessing system;

FIG. 3 depicts a schematic diagram of a single decontamination station of the reprocessing system of FIG. 2;

FIG. 4 depicts a schematic diagram of another single decontamination station of the reprocessing system of FIG. 2;

FIG. 5 a depicts a flow diagram of exemplary reprocessing cycles useable by the reprocessing system of FIG. 2; and

FIGS. 6A and 6B depict an exemplary method detailing exemplary steps of the cycles of FIG. 5.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Medical Device for Use with Reprocessing Apparatus

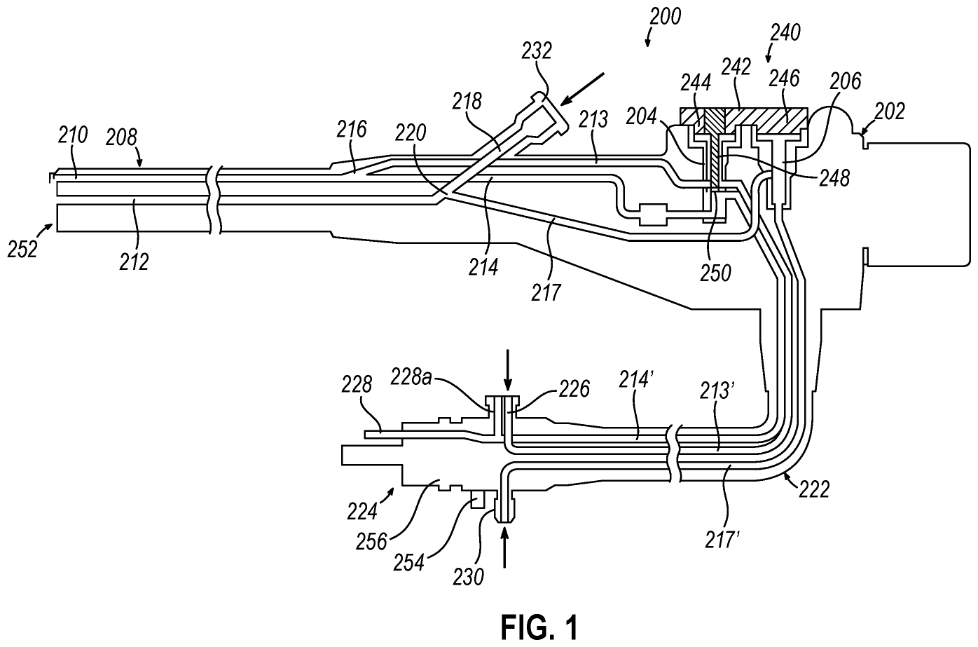

As shown in FIG. 1, endoscope (200) has a head part (202). Head part (202) includes openings (204, 206) formed therein. During normal use of endoscope (200), an air/water valve (not shown) and a suction valve (not shown) are arranged in openings (204, 206). A flexible shaft (208) is attached to head part (202). A combined air/water channel (210) and a combined suction/biopsy channel (212) are accommodated in shaft (208). A separate air channel (213) and water channel (214) are also arranged in head part (202) and merge into air/water channel (210) at the location of a joining point (216). Furthermore, a separate suction channel (217) and biopsy channel (218) are accommodated in head part (202) and merge into suction/biopsy channel (212) at the location of a joining point (220). It will be appreciated that the term “joining point” as used herein refers to an intersecting junction rather than being limited to a geometrical point and, the terms may be used interchangeably.

In head part (202), air channel (213) and water channel (214) open into opening (204) for the air/water valve (not shown). Suction channel (217) opens into opening (206) for the suction valve (not shown). Furthermore, a flexible feed hose (222) connects to head part (202) and accommodates channels (213′, 214′, 217′), which are connected to air channel (213), water channel (214), and suction channel (217) via respective openings (204, 206). In practice, feed hose (222) may also be referred to as the light-conductor casing. The mutually connecting air channels (213, 213′) will collectively be referred to below as air channel (213). The mutually connecting water channels (214, 214′) will collectively be referred to below as water channel (214). The mutually connecting suction channels (217, 217′) will collectively be referred to below as suction channel (217). A connection (226) for air channel (213), connections (228, 228a) for water channel (214), and a connection (230) for suction channel (217) are arranged on the end section (224) (also referred to as the light conductor connector) of flexible hose (222). When the connection (226) is in use, connection (228a) is closed off. A connection (232) for biopsy channel (218) is arranged on head part (202).

A channel separator (240) is shown inserted into openings (204, 206). Channel separator (240) comprises a body (242) and plug members (244, 246), which occlude respective openings (204, 206). A coaxial insert (248) on plug member (244) extends inwardly of opening (204) and terminates in an annular flange (250), which occludes a portion of opening (204) to separate channel (213) from channel (214). By connecting supply lines to openings (226, 228, 228a, 230, 232), liquid for cleaning and disinfection can be flowed through endoscope channels (213, 214, 217, 218) and out of a distal tip (252) of endoscope (200) via channels (210, 212). Channel separator (240) ensures that such liquid flows all the way through endoscope (200) without leaking out of openings (204, 206); and isolates channels (213, 214) from each other so that each channel (213, 214) has its own independent flow path. One of skill in the art will appreciate that various endoscopes having differing arrangements of channels and openings may require modifications to channel separator (240) to accommodate such differences while occluding ports in head (202) and keeping channels separated from each other so that each channel can be flushed independently of the other channels. Otherwise, a blockage in one channel might merely redirect flow to a connected unblocked channel.

A leakage port (254) on end section (224) leads into an interior portion (256) of endoscope (200) and is used to check for the physical integrity thereof, namely to ensure that no leakage has formed between any of the channels and the interior (256) or from the exterior to the interior (256).

II. Exemplary Medical Device Reprocessing System

In some instances, reprocessing systems use various chemicals and fluids to reprocess medical devices. Exposing certain components of the reprocessing system to such chemicals for long periods of time can cause damage to these components. In some instances, inert materials are used for those components subject to extended chemical exposure to combat the wear and damage that harsh chemicals can have on the components. In some instances, there is a need for service and/or repair and/or replacement of those components subjected to the chemicals. As described further below, an exemplary reprocessing system is configured and used in a way that addresses the effect of extended chemical exposure on the components of the reprocessing system.

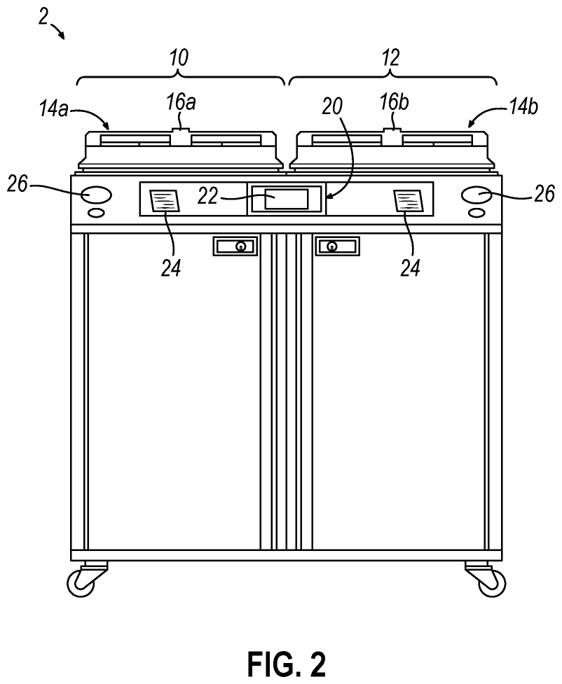

FIG. 2 shows an exemplary reprocessing system (2) that may be used to decontaminate endoscope (200) and other medical devices that include channels or lumens formed therethrough. System (2) of this example generally includes a first station (10) and a second station (12). Stations (10, 12) are at least substantially similar in all respects to provide for the decontamination of two different medical devices simultaneously or in series. First and second decontamination basins (14a, 14b) receive the contaminated devices. Each basin (14a, 14b) is selectively sealed by a respective lid (16a, 16b). In the present example, lids (16a, 16b) cooperate with respective basins (14a, 14b) to provide a microbe-blocking relationship to prevent the entrance of environmental microbes into basins (14a, 14b) during decontamination operations. By way of example only, lids (16a, 16b) may include a microbe removal or HEPA air filter formed therein for venting.

A control system or module (20) includes one or more microcontrollers, such as a programmable logic controller (PLC), for controlling decontamination and user interface operations. Although one control system (20) is shown herein as controlling both decontamination stations (10, 12), those skilled in the art will recognize that each station (10, 12) can include a dedicated control system. A visual display (22) displays decontamination parameters and machine conditions for an operator, and at least one printer (24) prints a hard copy output of the decontamination parameters for a record to be filed or attached to the decontaminated device or its storage packaging. It should be understood that printer (24) is merely optional. In some versions, visual display (22) is combined with a touch screen input device. In addition, or in the alternative, a keypad and/or other user input feature is provided for input of decontamination process parameters and for machine control. Other visual gauges (26) such as pressure meters and the like provide digital or analog output of decontamination or medical device leak testing data.

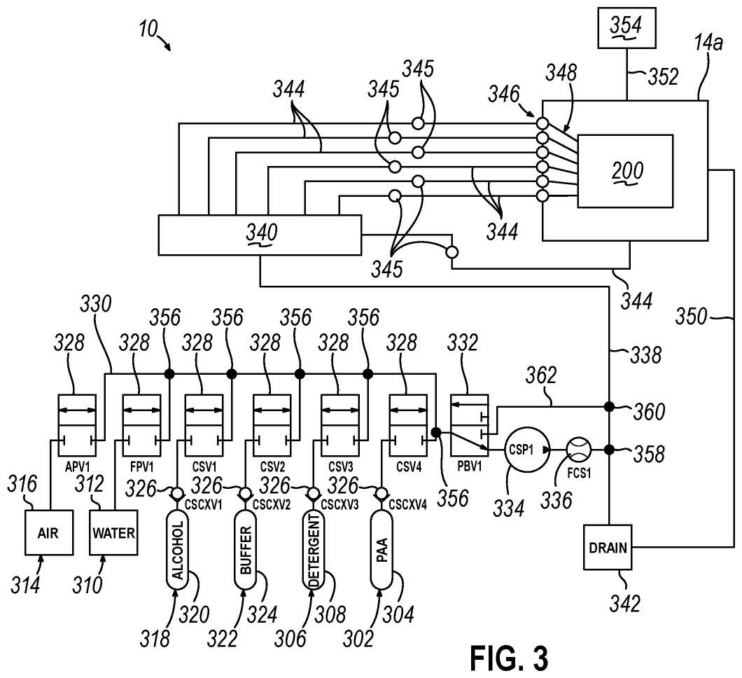

FIG. 3 diagrammatically illustrates one decontamination station (10) of reprocessing system (2), but those skilled in the art will recognize that decontamination station (12) may be configured and operable like decontamination station (10). It should also be understood that in some versions reprocessing system (2) may be provided with only a single decontamination station (10, 12) or more than two decontamination stations (10, 12).

Exemplary decontamination station (10) of reprocessing system (2) has a disinfectant source (302) with disinfectant (304) such as peracetic acid in the illustrated example, a detergent source (306) with detergent (308), a water source (310) with water (312), an air source (314) with air (316), an alcohol source (318) with alcohol (320), and a buffer source (322) with a buffer solution (324). In some examples, sources (302, 306, 310, 314, 318, 322) may be a storage tank or reservoir. In some examples water source (310) may be a connection to a private or public water supply, and air source (314) may be a connection to a sterilized or filtered air supply.

Each of disinfectant source (302), detergent source (306), alcohol source (318), and buffer source (322) is in fluid communication with a respective check valve (326). Check valves (326) permit fluid flow in a single direction—from the respective source (302, 306, 318, 322) to other components of reprocessor system (2). In this way, fluid is prevented from flowing from the other components of reprocessor system (2) back into any respective source (302, 306, 318, 322). While not shown in FIG. 3, water source (310) and air source (314) can optionally be coupled with a check valve in a similar manner.

After the respective check valves (326), each of disinfectant source (302), detergent source (306), alcohol source (318) and buffer source (322) is in fluid communication with a respective two-way valve (328). Additionally, each of water source (310) and air source (314) are in fluid communication with a respective two-way valve (328). Valves (328) are operable to open and close to either permit fluid flow or stop fluid flow. When a respective valve (328) is open such that fluid can flow, a pump (334) is operable to transfer the fluid from the respective source (302, 306, 310, 314, 318, 322) to a supply line (330), which is coupled with a three-way valve (332). Along supply line (330) are a plurality of two-way valves (356) that are operable to open and close to control fluid flow within supply line (330). In the present example, valves (328, 332, 356) are configured as solenoid valves, but other configurations for these valves may be used as will be apparent to those of ordinary skill in the art in view of the teachings herein.

Pump (334) is in fluid communication with valve (332), and further in fluid communication with a measuring device (336). In a configuration of valve (332) in an open state, pump (334) is able to transfer fluid from supply line (330) in the form of air (316), water (312), alcohol (320), buffer solution (324), detergent (308), and disinfectant, (304) from their respective sources (302, 306, 310, 314, 318, 322) to and through valve (332) and measuring device (336). In some versions, measuring device (336) is configured to determine volume flow of the fluid. For instance, measuring device (336) may comprise a flow meter of an impeller type or screw type. In some other versions, measuring device (336) is configured to determine a fluid flow time or duration. Still in some other versions, measuring device (336) is configured to determine both a volumetric flow rate as well as a flow time.

From measuring device (336), fluid is directed through a two-way valve (358) to a supply line (338) that fluidly connects with an inlet of a manifold (340) at one end and a drain (342) at an opposite end. As shown, supply line (338) also includes another two-way valve (360) that connects supply line (338) with a return line (362) leading back to valve (332). Fluid may be directed from measuring device (336) to manifold (340) or drain (342) depending on the position of valves (358, 360). For instance, a rinse and purge cycle or step may be completed as part of a reprocessing step to prepare pump (334) and measuring device (336) for a subsequent reprocessing step. In such steps, water (312) and/or air (316) can be directed to drain (342) after passing through pump (334) and measuring device (336). Alternatively, a washing, disinfecting, rinsing, or purging cycle may be completed as part of a reprocessing step to deliver fluid to and through manifold (340) to endoscope (200) within basin (14a).

A plurality of supply lines (344) couple with respective outlets of manifold (340). At an opposite end of supply lines (344), each respective supply line (344) couples with a respective one of a plurality of ports (346). Ports (346) are coupled with basin (14a) and provide access to an interior of basin (14a). Along respective supply lines (344), prior to ports (346), are valves (345). In the present example valves (345) are solenoid valves and are configured such that each valve (345) can be operated or actuated independently to permit fluid flow, stop fluid flow, or reduce or increase fluid flow. Within basin (14a) are a plurality of supply lines (348) that each couple with one of the respective ports (346) at one end. At the opposite end, each of the plurality of supply lines (348) couple with endoscope (200) within basin (14a) of reprocessing system (2). By connecting supply lines (348) to openings (226, 228, 228a, 230, 232) of endoscope (200) as described above, fluids for cleaning and disinfection can be flowed through endoscope channels (213, 214, 217, 218) and out of a distal tip (252) of endoscope (200) via channels (210, 212). In this manner, with valves (345) independently operable to control fluid flow from manifold (340), fluid can be selectively directed to channels (213, 214, 217, 218) of endoscope (200) depending on the state of valves (345). For instance, in some versions, fluid flow may be directed to one channel at a time. Still in other examples, fluid flow may be directed to multiple channels at a time. In view of the teachings herein, those of ordinary skill in the art will understand other ways to control valves (345) to direct fluid flow.

As shown, one of supply lines (344) couples with basin (14a) without a further connection to a channel of endoscope (200) to feed or direct fluid from manifold (340) to basin (14a). In this manner, fluid can be directed to the exterior surfaces of endoscope (200) within basin (14a). To aid in cleaning and disinfecting the exterior surface of endoscope (200), reprocessing system (2) can include one or more showers (not shown) and a recirculation pump (not shown) that draws fluid within basin (14a) and sprays the fluid over endoscope (200). As mentioned above, this dedicated supply line (344) for basin (14a) also includes one of valves (345) such that fluid flow can be selectively directed to basin (14a) independently from the fluid flow of other supply lines (344).

Basin (14a) couples with helical circulation tube (350) which permits fluid within basin (14a) to be directed to drain (342). This may include fluid collected within basin (14a) for cleaning and disinfecting the exterior surfaces of endoscope (200) as well as fluid flowed within and through channels (213, 214, 217, 218, 210, 212) of endoscope (200). Basin (14a) also couples with a supply line (352) that connects with a water supply (354) to supply water to basin (14a) for rinsing or dilution of other fluids added to basin (14a).

With this configuration for reprocessing system (2), by controlling valves (328, 356, 332, 358, 360), a single pump (334) and single measuring device (336) can be used to provide multiple fluids or chemicals to basin (14a), and ultimately endoscope (200) and its internal channels, for reprocessing. For instance, detergent (308) can be directed to basin (14a) and endoscope (200) in a wash step, followed by water (312) in a rinse step, followed by air (316) in a purge step, followed by disinfectant (304) in a disinfection step, followed by water (312) in a rinse step, followed by air (316) in a purge step. In this example, pump (334) and measuring device (336) are used and operable to deliver each of these fluids to basin (14a) and/or endoscope (200). Also, reprocessing system (2) is operable with rinsing and purging steps between chemical applications— such as washing with detergent and/or disinfecting with disinfectant—to avoid chemical to chemical interactions. Additionally, with the rinsing and purging steps, pump (334) and measuring device (336) are exposed to chemicals for a shorter time or duration. This prolongs the service life and reduces the wear on these components compared to systems where separate pumps and measuring devices would be exposed to a certain chemical for an extended time.

As shown in FIG. 3 alcohol source (318) and buffer source (322) are included to provide reprocessing system (2) with alcohol (320) and buffer solution (324) as needed. Alcohol (320) can be directed to endoscope (200) after a rinse step with water (312) and/or a purge step with air (316) to serve as a last step to displace water (312) and aid in drying endoscope (200). Buffer solution (324) can be directed to endoscope (200) in instances after detergent (308) or disinfectant (304) have been applied to return basin (14a) and/or endoscope (200) to a neutral or desired pH. In view of the teachings herein, other fluids and chemicals may be used with reprocessing system (2) instead of, or in addition to, those described above.

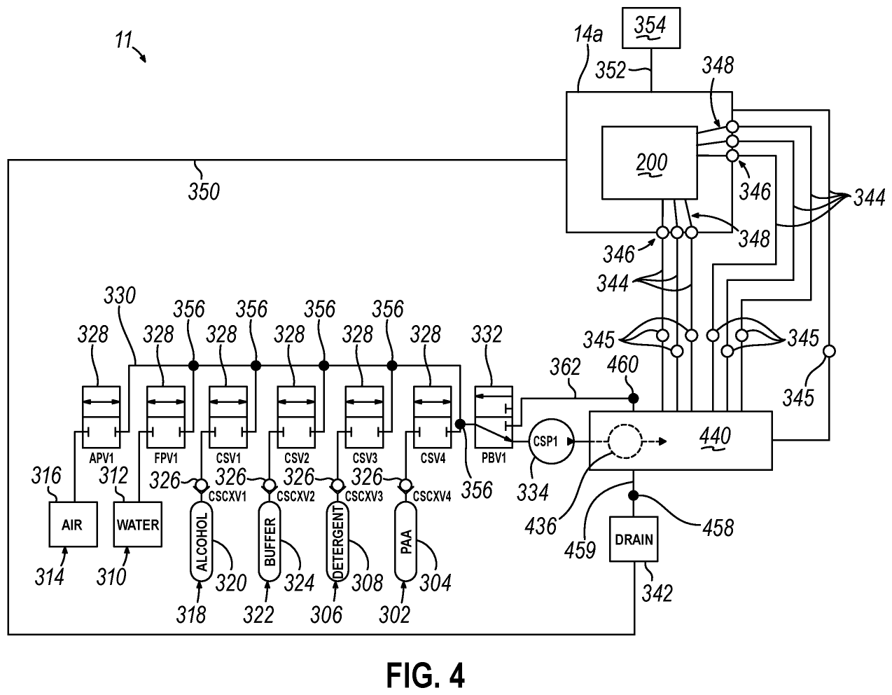

FIG. 4 illustrates another version of a decontamination station (11) of reprocessing system (2) that is similar to decontamination station (10) described above except that the measuring device is incorporated within the manifold. With decontamination station (11), air source (314), water source (310), alcohol source (318), buffer source (322), detergent source (306), and disinfectant source (302) are configured as described above with respect to decontamination station (10). Similarly, valves (328, 345, 356, 332) and pump (334) are configured as described above with respect to decontamination station (10).

With decontamination station (11), pump (334) is operable to direct fluid to manifold (440). Manifold (440) comprises measuring device (436), which is operable in the same manner as described above for measuring device (336) of decontamination station (10). After measuring device (336), fluid flows within and out from manifold (440) to supply lines (344) which carry fluid to valves (345) and to ports (346). Supply lines (344) couple with basin (14a) via ports (346) in the same manner as described above with respect to decontamination station (10). Similarly, with decontamination station (11) the connections from ports (346) to supply lines (348) to endoscope (200) are the same as described above with respect to decontamination station (10).

With decontamination station (11), manifold (440) also couples with drain line (459) that includes a valve (458) to control flow to drain (342). Fluid may be flowed to drain (342), for example, with a rinse and purge cycle or step that may be completed as part of a reprocessing step to prepare pump (334) and measuring device (436) for a subsequent reprocessing step. In such steps, water (312) and/or air (316) can be directed to drain (342) after passing through pump (334) and measuring device (436) within manifold (440). Also, similar to decontamination station (10), decontamination station (11) includes return line (362) that couples valve (332) with valve (460) and thereafter manifold (440).

With respect to decontamination stations (10, 11) described above, the electrical and electromechanical devices shown and described are operatively connected to and controlled by control system (20). Specifically, inputs are provided to control system (20), which controls the cleaning and/or disinfection cycles and other machine operations in accordance therewith. Such inputs can include, among other things, the position of various valves and the values determined by measuring device (336, 436). For example, based on the inputs, control system (20) includes outputs that are operatively connected to pump (334) and valves (326, 328, 345, 356, 332, 358, 360, 458, 460) to control these devices for effective cleaning and/or disinfection cycles and other operations. Other exemplary electrical and electromechanical devices may be used with reprocessing system (2), some of which are described in U.S. Pat. No. 10,792,386 entitled, “Apparatus and Method to Repeatedly Fill and Purge Channels of Endoscope,” issued Oct. 6, 2020, which is incorporated by reference herein.

III. Exemplary Medical Device Reprocessing Method

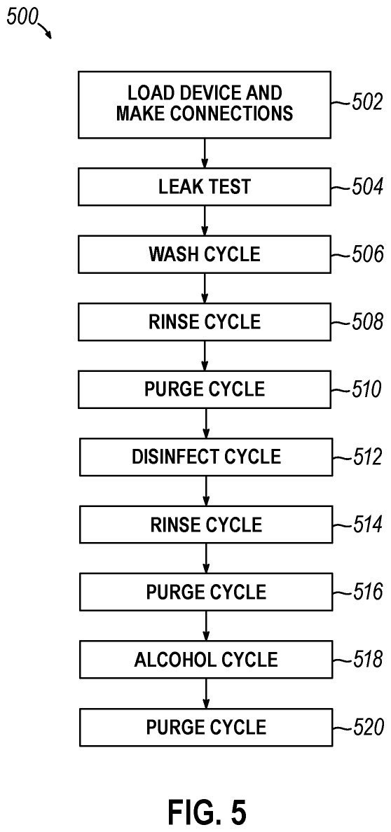

Referring to FIG. 5, in an exemplary method of use of reprocessing system (2), an operator may start reprocessing method (500) loading endoscope (200) into reprocessor system (2) and making connections (502). To do so, the operator may actuate a foot pedal (not shown) to open basin lid (16a). Each lid (16a, 16b) may have its own foot pedal. In some versions, once pressure is removed from the foot pedal, the motion of lid (16a, 16b) stops. With lid (16a) open, the operator inserts shaft (208) of endoscope (200) into helical circulation tube (350). End section (224) and head section (202) of endoscope (200) are situated within basin (14a), with feed hose (222) coiled within basin (14a) with as wide a diameter as possible. Next, supply lines (348) are attached to respective endoscope openings (226, 228, 228a, 230, 232). In some versions, supply lines (348) are color coded, and a guide located on the decontamination station provides a reference for the color-coded connections.

Depending on the customer-selectable configuration, control system (20) may prompt the operator to enter a user code, patient ID, endoscope code, and/or specialist code. This information may be entered manually (e.g., through touch screen (22)), automatically (e.g., by using an attached barcode wand), or in any other suitable fashion. With the information entered (if required), the operator may then close lid (16a). In some versions, closing lid (16a) requires the operator to press a hardware button and a touch-screen (22) button simultaneously to provide a fail-safe mechanism for preventing the operator's hands from being caught or pinched by the closing basin lid (16a). If either the hardware button or software button is released while lid (16a) is in the process of closing, the motion of lid (16a) stops.

Once lid (16a) is closed, a leak test occurs (504). To initiate leak test (504), the operator may press a button on touch-screen (22). At the start of leak test (504), pump (334) is activated and valve (328) associated with air source (314) is opened to deliver air (316) within the body of endoscope (200) to monitor pressure for leaks. When pressure reaches a predetermined level (e.g., 250 mbar), pump (334) is deactivated, and the pressure is allowed to stabilize for a certain stabilization period (e.g., 6 seconds). If pressure has not reached a certain pressure (e.g., 250 mbar) in a certain time period (e.g., 45 seconds), the program is stopped and the operator is notified of a leak. If pressure drops below a threshold (e.g., less than 100 mbar) during the stabilization period, the program is stopped and the operator is notified of the condition. Once the pressure has stabilized, the pressure drop is monitored over the course of a certain duration (e.g., 60 seconds). If the pressure drop is faster than a predetermined rate (e.g., more than 10 mbar within 60 seconds), the program is stopped and the operator is notified of the condition. If the pressure drop is slower than a predetermined rate (e.g., less than 10 mbar in 60 seconds), reprocessing system (2) continues with the next step. A slight positive pressure is held within the body of endoscope (200) during the rest of the process to prevent fluids from leaking in.

Leak test (504) also includes checking the adequacy of connection to the various ports (226, 228, 228a, 230, 232) and the proper placement of channel separator (240). To do this, a quantity of water is admitted to basin (14a) from water supply (354) to submerge the distal end of endoscope (200) in helical tube (350). Valve (332) is controlled to direct fluid through return line (362) to pump (334). Valves (360, 460) are opened and pump (334) is run in reverse to draw a vacuum and to ultimately draw liquid into endoscope channels (210, 212). Pressure sensors (not shown) are monitored to make sure that the pressure in any one channel (210, 212) does not drop and/or raise by more than a predetermined amount in a given time frame. If it does, it likely indicates that one of the connections was not made correctly and air is leaking into channel (210, 212). In any event, in the presence of an unacceptable pressure drop, control system (20) will indicate a likely faulty connection, preferably with an indication of which channel (210, 212) failed.

In the event that the leak tests are passed, reprocessing method (500) proceeds to a wash cycle (506). In some versions, an optional pre-rinse is carried out prior to wash cycle (560) or as the first part of wash cycle (506). With the optional pre-rinse step, water is flushed through channels (210, 212, 213, 214, 217, 218) to remove waste material prior to washing with detergent (308). With the pre-rinse step, basin (14a) is filled with water from water supply (354). Similar to leak test (504), water is drawn through interior of channels (210, 212, 213, 214, 217, 218) by pump (334) and directed to drain (342). As the water is being pumped through channels (210, 212, 213, 214, 217, 218) to drain (342), basin (14a) is emptied.

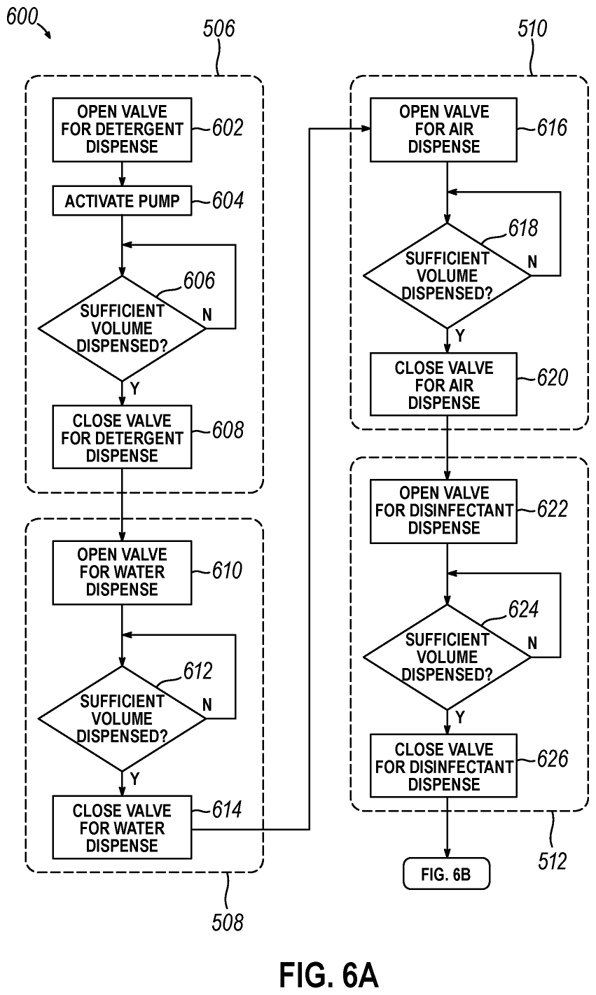

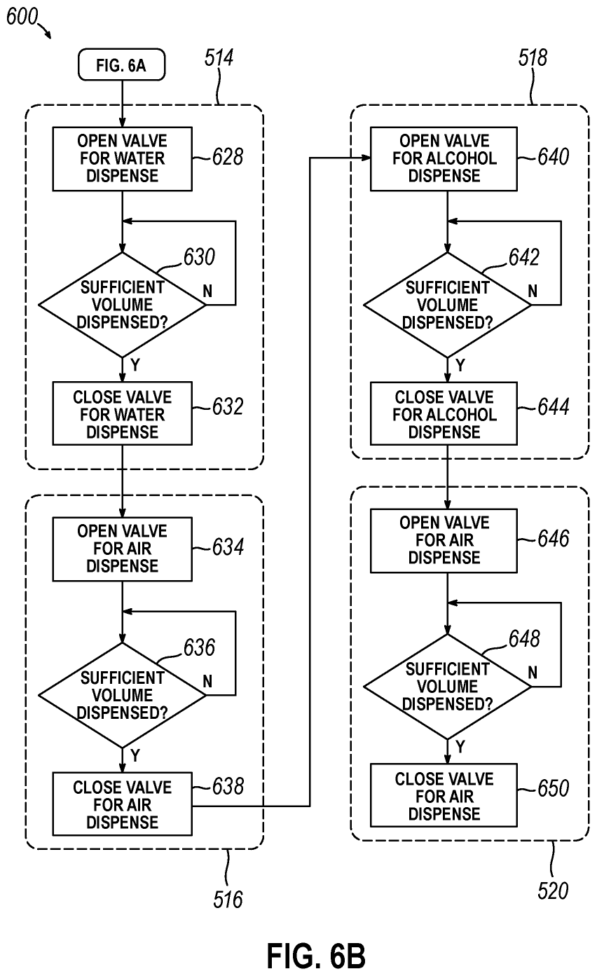

Once the optional pre-rinse step is complete, wash cycle (506) begins or continues by filling basin (14a) with water from water supply (354). In some versions, the water may be heated to warm the water to e.g., approximately 35° C. Referring now also to FIGS. 6A and 6B, which shows a schematic diagram of a method (600) used when adding detergent (308) to basin (14a) and endoscope (200), reprocessing system (2) adds enzymatic detergent (308) to the water in basin (14a) and to the internal endoscope channels (210, 212, 213, 214, 217, 218) by means of pump (334) and valve (328) associated with detergent source (306) to send detergent (308) to supply lines (344). In doing so, valve (328) associated with detergent source (306) is first opened (602). Thereafter pump (334) is activated (604) which begins the flow of detergent (308).

The volume of added detergent (308) is controlled by control system (20) based on input from measuring device (336, 436) that determines the volume flow and/or the flow time of the fluid. This information is provided to control system (20), which uses this information to calculate the volume of added or dispensed detergent (308). Control system (20) includes logic that compares the volume of dispensed detergent (308) with a predetermined, target, or threshold volume of detergent (308) to determine if the dispensed volume of detergent (308) is sufficient based on the dispensed volume meeting the predetermined volume (606). Where the sufficiency in volume is not yet met, valve (328) associated with detergent source (306) remains open and pump (334) activated to continue dispensing detergent (308). Once the volume of dispensed detergent (308) is sufficient, control system (20) closes valve (328) associated with detergent source (306) (608) to stop the flow or dispensing of detergent (308).

With wash cycle (506), instead of controlling detergent dispensing based on volume as described above, in other versions, control system (20) may control the detergent dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with detergent source (306) accordingly.

In the present example, with wash cycle (506), reprocessing system (2) directs fluid to drain (342) via tube (350) as needed to maintain a target or predetermined fluid level within basin (14a). Additionally, at the completion of wash cycle (506), control system (20) directs fluid within basin (14a) and endoscope (200) to drain (342) via tube (350) in preparation for subsequent cycles as shown in FIG. 5. Draining fluid from basin (14a) and channels (210, 212, 214, 214, 217, 218) can be done by gravity or with the assistance of a drain pump (not shown).

After wash cycle (506), according to FIG. 5, a rinse cycle (508) is performed. With rinse cycle (508), referring to FIGS. 6A and 6B, control system (20) opens valve (328) associated with water source (310) (610) to rinse basin (14a) and endoscope (200) with water (312). With pump (334) activated, this action directs rinse water (312) to basin (14a) and to the internal endoscope channels (210, 212, 213, 214, 217, 218). In the present example, pump (334) remains activated while valves (328) associated with detergent source (306) and water source (310) are actuated. However, this is not required in all versions. For instance, in some versions, pump (334) may be deactivated while valves (328) associated with detergent source (306) and water source (310) are actuated, and then pump (334) reactivated.

The volume of added water (312) is controlled by control system (20) based on input from measuring device (336, 436) that determines the volume flow and/or the flow time of the fluid. This information is provided to control system (20), which uses this information to calculate the volume of added or dispensed water (312). Control system (20) includes logic that compares the volume of dispensed water (312) with a predetermined, target, or threshold volume of water (312) to determine if the dispensed volume of water (312) is sufficient based on the dispensed volume meeting the predetermined volume (612). Where the sufficiency in volume is not yet met, valve (328) associated with water source (310) remains open and pump (334) activated to continue dispensing water (312). Once the volume of dispensed water (312) is sufficient, control system (20) closes valve (328) associated with water source (310) (614) to stop the flow or dispensing of water (312).

With rinse cycle (508), instead of controlling water dispensing based on volume as described above, in other versions, control system (20) may control the water dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with water source (310) accordingly.

In the present example, with rinse cycle (508), reprocessing system (2) directs fluid to drain (342) via tube (350) as needed to maintain a target or predetermined fluid level within basin (14a). Additionally, at the completion of rinse cycle (508), control system (20) directs fluid within basin (14a) and endoscope (200) to drain (342) via tube (350) in preparation for subsequent cycles as shown in FIG. 5. Draining fluid from basin (14a) and channels (210, 212, 214, 214, 217, 218) can be done by gravity or with the assistance of a drain pump (not shown).

After rinse cycle (508), according to FIG. 5, a purge cycle (510) is performed. With purge cycle (510), referring to FIGS. 6A and 6B, control system (20) opens valve (328) associated with air source (314) (616) to purge internal endoscope channels (210, 212, 213, 214, 217, 218) of water (312) from rinse cycle (508). With pump (334) activated, air (316) is directed to internal endoscope channels (210, 212, 213, 214, 217, 218). In the present example, pump (334) remains activated while valves (328) associated with water source (310) and air source (314) are actuated. However, this is not required in all versions. For instance, in some versions, pump (334) may be deactivated while valves (328) associated with water source (310) and air source (314) are actuated, and then pump (334) reactivated.

The volume of added air (316) is controlled by control system (20) based on input from measuring device (336, 436) that determines the volume flow and/or the flow time of the fluid. This information is provided to control system (20), which uses this information to calculate the volume of added or dispensed air (316). Control system (20) includes logic that compares the volume of dispensed air (316) with a predetermined, target, or threshold volume of air (316) to determine if the dispensed volume of air (316) is sufficient based on the dispensed volume meeting the predetermined volume (618). Where the sufficiency in volume is not yet met, valve (328) associated with air source (314) remains open and pump (334) activated to continue dispensing air (316). Once the volume of dispensed air (316) is sufficient, control system (20) closes valve (328) associated with air source (314) (620) to stop the flow or dispensing of air (316).

With purge cycle (510), instead of controlling air dispensing based on volume as described above, in other versions, control system (20) may control the air dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with air source (314) accordingly.

Thereafter, according to FIG. 5, a disinfect cycle (512) is performed. Disinfect or disinfectant cycle (512) begins or continues by filling basin (14a) with water from water supply (354). In some versions, the water may be heated to warm the water to e.g., approximately 35° C. Referring now also to FIGS. 6A and 6B, which shows a schematic diagram of a method (600) used when adding disinfectant (304) to basin (14a) and endoscope (200), reprocessing system (2) adds disinfectant (304) to the water in basin (14a) and to the internal endoscope channels (210, 212, 213, 214, 217, 218) by means of pump (334) and valve (328) associated with disinfectant source (302) to send disinfectant (304) to supply lines (344). Valve (328) associated with disinfectant source (302) is first opened (622). With pump (334) previously activated (604), the flow of disinfectant (304) begins.

In the present example, disinfectant (304) is delivered to endoscope channels (210, 212, 213, 214, 217, 218) in its same concentration as disinfectant (304) from disinfectant source (302). With water from water supply (354) added to basin (14a), disinfectant (304) directed to basin (14a) is diluted to a lower concentration compared to the concentration of disinfectant (304) delivered to endoscope internal channels (210, 212, 213, 214, 217, 218). In other versions, the concentration of disinfectant (304) delivered to internal channels (210, 212, 213, 214, 217, 218) and to the exterior of endoscope (200) within basin (14a) can be the same or about the same. For instance, dilution water from water supply (354) may not be provided to basin (14a) in some versions, Also by way of example only, dilution water from water supply (354) can be added to supply lines (344) before supply lines (344) reach ports (346). In view of the teachings herein, other ways to control dilution of disinfectant (304) and/or detergent (308) will be apparent to those of ordinary skill in the art.

The volume of added disinfectant (304) is controlled by control system (20) based on input from measuring device (336, 436) that determines the volume flow and/or the flow time of the fluid. This information is provided to control system (20), which uses this information to calculate the volume of added or dispensed disinfectant (304). Control system (20) includes logic that compares the volume of dispensed disinfectant (304) with a predetermined, target, or threshold volume of disinfectant (304) to determine if the dispensed volume of disinfectant (304) is sufficient based on the dispensed volume meeting the predetermined volume (624). Where the sufficiency in volume is not yet met, valve (328) associated with disinfectant source (302) remains open and pump (334) activated to continue dispensing disinfectant (304). Once the volume of dispensed disinfectant (304) is sufficient, control system (20) closes valve (328) associated with disinfectant source (302) (626) to stop the flow or dispensing of disinfectant (304).

With disinfect cycle (512), instead of controlling disinfectant dispensing based on volume as described above, in other versions, control system (20) may control the disinfectant dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with disinfectant source (302) accordingly.

In the present example, with disinfect cycle (512), reprocessing system (2) directs fluid to drain (342) via tube (350) as needed to maintain a target or predetermined fluid level within basin (14a). Additionally, at the completion of disinfect cycle (512), control system (20) directs fluid within basin (14a) and endoscope (200) to drain (342) via tube (350) in preparation for subsequent cycles as shown in FIG. 5. Draining fluid from basin (14a) and channels (210, 212, 214, 214, 217, 218) can be done by gravity or with the assistance of a drain pump (not shown).

After disinfect cycle (512), according to FIG. 5, a rinse cycle (514) is performed. With rinse cycle (514), referring to FIG. 6B, control system (20) opens valve (328) associated with water source (310) (628) to rinse basin (14a) and endoscope (200) with water (312). With pump (334) activated, this action directs rinse water (312) to basin (14a) and to the internal endoscope channels (210, 212, 213, 214, 217, 218). In the present example, pump (334) remains activated while valves (328) associated with disinfectant source (302) and water source (310) are actuated. However, this is not required in all versions. For instance, in some versions, pump (334) may be deactivated while valves (328) associated with disinfectant source (302) and water source (310) are actuated, and then pump (334) reactivated.

The volume of added water (312) is controlled by control system (20) based on input from measuring device (336, 436) that determines the volume flow and/or the flow time of the fluid. This information is provided to control system (20), which uses this information to calculate the volume of added or dispensed water (312). Control system (20) includes logic that compares the volume of dispensed water (312) with a predetermined, target, or threshold volume of water (312) to determine if the dispensed volume of water (312) is sufficient based on the dispensed volume meeting the predetermined volume (630). Where the sufficiency in volume is not yet met, valve (328) associated with water source (310) remains open and pump (334) activated to continue dispensing water (312). Once the volume of dispensed water (312) is sufficient, control system (20) closes valve (328) associated with water source (310) (632) to stop the flow or dispensing of water (312).

With rinse cycle (514), instead of controlling water dispensing based on volume as described above, in other versions, control system (20) may control the water dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with water source (310) accordingly.

In the present example, with rinse cycle (514), reprocessing system (2) directs fluid to drain (342) via tube (350) as needed to maintain a target or predetermined fluid level within basin (14a). Additionally, at the completion of rinse cycle (514), control system (20) directs fluid within basin (14a) and endoscope (200) to drain (342) via tube (350) in preparation for subsequent cycles as shown in FIG. 5. Draining fluid from basin (14a) and channels (210, 212, 214, 214, 217, 218) can be done by gravity or with the assistance of a drain pump (not shown).

After rinse cycle (514), according to FIG. 5, a purge cycle (516) is performed. With purge cycle (516), referring to FIG. 6B, control system (20) opens valve (328) associated with air source (314) (640) to purge internal endoscope channels (210, 212, 213, 214, 217, 218) of water (312) from rinse cycle (514). With pump (334) activated, air (316) is directed to internal endoscope channels (210, 212, 213, 214, 217, 218). In the present example, pump (334) remains activated while valves (328) associated with water source (310) and air source (314) are actuated. However, this is not required in all versions. For instance, in some versions, pump (334) may be deactivated while valves (328) associated with water source (310) and air source (314) are actuated, and then pump (334) reactivated.

The volume of added air (316) is controlled by control system (20) based on input from measuring device (336, 436) that determines the volume flow and/or the flow time of the fluid. This information is provided to control system (20), which uses this information to calculate the volume of added or dispensed air (316). Control system (20) includes logic that compares the volume of dispensed air (316) with a predetermined, target, or threshold volume of air (316) to determine if the dispensed volume of air (316) is sufficient based on the dispensed volume meeting the predetermined volume (636). Where the sufficiency in volume is not yet met, valve (328) associated with air source (314) remains open and pump (334) activated to continue dispensing air (316). Once the volume of dispensed air (316) is sufficient, control system (20) closes valve (328) associated with air source (314) (638) to stop the flow or dispensing of air (316).

With purge cycle (516), instead of controlling air dispensing based on volume as described above, in other versions, control system (20) may control the air dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with air source (314) accordingly.

Thereafter, according to FIG. 5, an optional alcohol cycle (518) is performed. With alcohol cycle (518), referring to FIG. 6B, control system (20) opens valve (328) associated with alcohol source (218) (640) to rinse basin (14a) and endoscope (200) with alcohol (320). With pump (334) activated, this action directs alcohol (320) to basin (14a) and to the internal endoscope channels (210, 212, 213, 214, 217, 218). In the present example, pump (334) remains activated while valves (328) associated with alcohol source (318) and water source (310) are actuated. However, this is not required in all versions. For instance, in some versions, pump (334) may be deactivated while valves (328) associated with alcohol source (318) and water source (310) are actuated, and then pump (334) reactivated.

The volume of added alcohol (320) is controlled by control system (20) based on input from measuring device (336, 436) that determines the volume flow and/or the flow time of the fluid. This information is provided to control system (20), which uses this information to calculate the volume of added or dispensed alcohol (320). Control system (20) includes logic that compares the volume of dispensed alcohol (320) with a predetermined, target, or threshold volume of alcohol (320) to determine if the dispensed volume of alcohol (320) is sufficient based on the dispensed volume meeting the predetermined volume (642). Where the sufficiency in volume is not yet met, valve (328) associated with alcohol source (318) remains open and pump (334) activated to continue dispensing alcohol (320). Once the volume of dispensed alcohol (320) is sufficient, control system (20) closes valve (328) associated with alcohol source (318) (644) to stop the flow or dispensing of alcohol (320).

With alcohol cycle (518), instead of controlling alcohol dispensing based on volume as described above, in other versions, control system (20) may control the alcohol dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with alcohol source (318) accordingly.

Thereafter, according to FIG. 5, a purge cycle (520) is performed. With purge cycle (520), referring to FIG. 6B, control system (20) opens valve (328) associated with air source (314) (646) to purge internal endoscope channels (210, 212, 213, 214, 217, 218) of alcohol (320) from alcohol cycle (518). With pump (334) activated, air (316) is directed to internal endoscope channels (210, 212, 213, 214, 217, 218). In the present example, pump (334) remains activated while valves (328) associated with alcohol source (318) and air source (314) are actuated. However, this is not required in all versions. For instance, in some versions, pump (334) may be deactivated while valves (328) associated with alcohol source (318) and air source (314) are actuated, and then pump (334) reactivated.

The volume of added air (316) is controlled by control system (20) based on input from measuring device (336, 436) that determines the volume flow and/or the flow time of the fluid. This information is provided to control system (20), which uses this information to calculate the volume of added or dispensed air (316). Control system (20) includes logic that compares the volume of dispensed air (316) with a predetermined, target, or threshold volume of air (316) to determine if the dispensed volume of air (316) is sufficient based on the dispensed volume meeting the predetermined volume (648). Where the sufficiency in volume is not yet met, valve (328) associated with air source (314) remains open and pump (334) activated to continue dispensing air (316). Once the volume of dispensed air (316) is sufficient, control system (20) closes valve (328) associated with air source (314) (650) to stop the flow or dispensing of air (316).

With purge cycle (520), instead of controlling air dispensing based on volume as described above, in other versions, control system (20) may control the air dispensing based on flow time. In such versions, the predetermined metric is time of flow as opposed to a volume of flow. For instance, measuring device (336, 436) measures flow time and provides such information to control system (20) which assesses the sufficiency of the flow time and then actuates valve (328) associated with air source (314) accordingly.

After the conclusion of either purge cycle (516) or purge cycle (520)—depending whether optional alcohol cycle (518) was performed—endoscope (200) reprocessing is complete and endoscope (200) ready for unloading from reprocessing system (2) for subsequent use. Note that in some examples of methods (500, 600) drying cycles may be included where sterilized air is blown over the exterior of endoscope (200) and/or through internal channels (210, 212, 213, 214, 217, 218). At least some such drying steps or processes that can be used with the systems and methods described herein are described in PCT Patent App. No. PCT/US20/36254, entitled “System and Method for Drying Channels of Medical Instrument During Cleaning,” filed Jun. 5, 2020, the disclosure of which is incorporated by reference herein. Still, other reprocessing methods to which channel drying can be used will be apparent to those of ordinary skill in the art in view of the teachings herein.

Additionally, in some examples of reprocessing system (2) and methods (500, 600), one or more buffer cycles can be included to pass a neutralizing fluid over and/or through endoscope (200) and its channels (210, 212, 213, 214, 217, 218). This may be the case where detergent (308) or disinfectant (304) may have a basic or acidic pH and it is desirable flush endoscope (200) with a neutralizing fluid or buffer solution (324) prior to performing subsequent reprocessing steps.

Referring to FIGS. 5-6B, the cycles (506, 508, 510, 512, 514, 516, 518, 520) can be repeated in the sequence shown or in any other sequence, including partial sequences for a predetermined number of times. For instance, in one version of method (500), after purge cycle (516), wash cycle (506) is repeated followed by rinse cycle (508), purge cycle (510), disinfect cycle (512), rinse cycle (514), purge cycle (516), alcohol cycle (518), and purge cycle (520). In another version of method (500), disinfect cycle (512), rinse cycle (514), and purge cycle (516) are repeated a predetermined number of times before alcohol cycle (518) is conducted. Similarly, in some versions, wash cycle (506), rinse cycle (508), and purge cycle (510) are repeated a predetermined number of times prior to beginning disinfect cycle (512). In view of the teachings herein, repeats of other partial sequences of the cycles illustrated in FIG. 5 will be apparent to those of ordinary skill in the art.

In some versions, after the final purge cycle (516, 518), reprocessing system (2) begins a final leak test. In particular, reprocessing system (2) pressurizes the body of endoscope (200) and measures the leak rate as described above. If the final leak test is successful, reprocessing system (2) indicates the successful completion of the cycles by displaying a cycle complete indication on a graphical user interface (GUI), e.g., via touch-screen (22). From the time of program completion to the time at which lid (16a) is opened, pressure within the body of endoscope (200) is normalized to atmospheric pressure.

Depending on customer-selected configuration, reprocessing system (2) may prevent lid (16a) from being opened until receiving user confirmation, e.g., a valid user identification code may be entered to provide user confirmation. Information about the completed program, including the user ID, endoscope ID, specialist ID, and patient ID are stored along with the sensor data obtained throughout the program. If a printer is connected to reprocessing system (2), and if requested by the operator, a record of the disinfection program will be printed. Once user confirmation has been received, lid (16a) may be unlocked and opened (e.g., using the foot pedal as described above). Endoscope (200) is then removed where endoscope (200) is disconnected from supply lines (348) and removed from basin (14a). Lid (16a) can then be closed using both the hardware and software buttons as described above. With device (200) removed from reprocessing system (2), reprocessing methods (500, 600) end and may be repeated thereafter for reprocessing additional devices, i.e. endoscopes (200).

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus for reprocessing an internal channel of a medical device comprises (a) a port configured to couple with the internal channel of the medical device to provide access to the internal channel of the medical device; (b) a measuring device coupled with the port, wherein the measuring device is configured to determine a flow volume; (c) a pump coupled with the measuring device, wherein the pump is in fluid communication with a plurality of fluid sources, each of the fluid sources in fluid communication with a respective fluid, wherein the pump is configured to deliver the respective fluid from the fluid source to the port through the measuring device; (d) a first plurality of valves, wherein each valve is in fluid communication the pump and also in fluid communication with one of the plurality of fluid sources; and (e) a control module operable to (i) open the first plurality of valves, (ii) close the first plurality of valves, (iii) activate the pump to deliver the respective fluid from each of the fluid sources through a select one of the first plurality of valves to the pump and through the measuring device to the port, and (vi) deactivate the pump to terminate delivery of the respective fluid from each of the fluid sources to the port.

### Example 2

The apparatus of Example 1, wherein the measuring device comprises a flow meter.

### Example 3

The apparatus of any one or more of Example 1 through Example 2, wherein the measuring device is operable to determine a flow time.

### Example 4

The apparatus of any one or more of Example 1, through Example 3, wherein the plurality of fluid sources comprises a detergent source in fluid communication with a detergent, a water source in fluid communication with a water, a pressurized air source in fluid communication with a pressurized air, and a disinfectant source in fluid communication with a disinfectant.

### Example 5

The apparatus of Example 4, wherein the control module is operable to sequentially deliver to the port: (a) the detergent from the detergent source, (b) the water from the water source, and (c) the pressurized air from the pressurized air source.

### Example 6

The apparatus of any one or more of Example 4 through Example 5, wherein the control module is operable to sequentially deliver to the port: (d) the disinfectant from the disinfectant source, (e) the water from the water source, and (f) the pressurized air from the pressurized air source.

### Example 7

The apparatus of any one or more of Example 1 through Example 6, wherein the plurality of fluid sources further comprises a buffer solution source in fluid communication with a buffer solution, wherein the control module is operable to deliver to the port the buffer solution from the buffer solution source.

### Example 8

The apparatus of any one or more of Example 1 through Example 7, wherein the plurality of fluid sources further comprises an alcohol source in fluid communication with an alcohol, wherein the control module is operable to deliver to the port the alcohol from the alcohol source.

### Example 9

The apparatus of any one or more of Example 1 through Example 8, wherein the control module is operable for a predetermined number of cycles.

### Example 10

The apparatus of any one or more of Example 1 through Example 9, wherein the control module is operable to repeat sequential delivery of the disinfectant and the pressurized air to the port for a predetermined number of cycles.

### Example 11

The apparatus of any one or more of Example 1 through Example 10, wherein the control module is operable to rinse the pump and the measuring device with the water and then purge the pump and the measuring device of the water using the pressurized air between each delivery to the port of the detergent and the disinfectant.

### Example 12

The apparatus of any one or more of Example 1 through Example 11, further comprising a manifold that couples the measuring device with the port.

### Example 13

The apparatus of any one or more of Example 1 through Example 12, wherein the medical device comprises multiple internal channels, and wherein the apparatus comprises multiple ports that each couple with a respective one of the multiple internal channels of the medical device.

### Example 14

The apparatus of Example 13, wherein the measuring device is coupled with the multiple ports via the manifold.

### Example 15

The apparatus of any of Example 12 through Example 14, further comprising a second plurality of valves, wherein each valve of the second plurality of valves is in fluid communication with the manifold and also in fluid communication with a respective one of the multiple internal channels of the medical device, wherein each valve of the second plurality of valves is independently operable to selectively control fluid flow between the manifold and the respective one of the multiple channels of the medical device.

### Example 16

An apparatus for reprocessing an internal channel of a medical device comprises (a) a port configured to couple with the internal channel of the medical device and configured to direct a fluid to the internal channel of the medical device. The fluid is a detergent, a water, a pressurized air, or a disinfectant. The apparatus also comprises (b) a measuring device coupled with the port. The measuring device is configured to determine at least a select one of a flow volume and a flow time. The apparatus also comprises (c) a pump coupled with the measuring device, wherein the pump is in fluid communication with a detergent source, a water source, a pressurized air source, and a disinfectant source. The pump is configured to deliver the detergent from the detergent source to the port through the measuring device. The pump is further configured to deliver the water from the water source to the port through the measuring device. The pump is further configured to deliver the pressurized air from the pressurized air source to the port through the measuring device. The pump is further configured to deliver the disinfectant from the disinfectant source to the port through the measuring device. The apparatus also comprises (d) a plurality of valves, wherein each valve is in fluid communication with the pump and also in fluid communication with one of the detergent source, the water source, the pressurized air source, and the disinfectant source. The apparatus also comprises (e) a control module operable to control the plurality of valves to open and close the plurality of valves. The control module is operable to: (i) activate the pump to deliver the detergent from the detergent source through a select one of the plurality of valves to the pump and through the measuring device to the port, (ii) terminate delivery of the detergent in response to a select one of a predetermined time limit for detergent fluid flow and a flow volume of the detergent as determined by the measuring device, (iii) activate the pump to deliver the water from the water source through a select one of the plurality of valves to the pump and through the measuring device to the port, (iv) terminate delivery of the water in response to a select one of a predetermined time limit for water fluid flow and a flow volume of the water as determined by the measuring device, (v) activate the pump to deliver the pressurized air from the pressurized air source through a select one of the plurality of valves to the pump and through the measuring device to the port, (vi) terminate delivery of the pressurized air in response to a select one of a predetermined time limit for pressurized air fluid flow and a flow volume of the pressurized air as determined by the measuring device, (vii) activate the pump to deliver the disinfectant from the disinfectant source through a select one of the plurality of valves to the pump and through the measuring device to the port, and (viii) terminate delivery of the disinfectant in response to a select one of a predetermined time limit for disinfectant fluid flow and a flow volume of the disinfectant as determined by the measuring device.

### Example 17

The apparatus of Example 16, wherein the control module is operable to repeat the delivery of the disinfectant and the pressurized air and from the pump to the port for a predetermined number of cycles

### Example 18

A method of reprocessing a medical device having one or more channels comprises: (a) loading the device having the one or more channels into a reprocessing system comprising: (i) one or more ports, (ii) a manifold coupled with the one or more ports, (iii) a measuring device coupled with the manifold, (iv) a pump coupled with the measuring device, (v) a detergent source in fluid communication with a detergent, (vi) a water source in fluid communication with a water, (vii) a pressurized air source in fluid communication with a pressurized air, (viii) a disinfectant source in fluid communication with a disinfectant, (vi) a plurality of first valves each in fluid communication with the pump and a respective one of the detergent source, the water source, the pressurized air source, and the disinfectant source, and (vii) a control module operable to control the pump and the plurality of first valves; (b) connecting each of the one or more channels of the device with the one or more ports; (c) conducting a wash cycle using the control module of the reprocessing system, comprising: (i) opening the valve of the plurality of first valves in fluid communication with the detergent source, (ii) activating the pump to deliver detergent to the one or more channels via the measuring device and the one or more ports, and (iii) closing the valve of the plurality of first valves in fluid communication with the detergent source in response to reaching either a predetermined detergent flow volume or a predetermined detergent flow time as determined by the measuring device; (d) conducting a rinse cycle using the control module of the reprocessing system, comprising: (i) opening the valve of the plurality of first valves in fluid communication with the water source, (ii) activating the pump to deliver water to the one or more channels via the measuring device and the one or more ports, and (iii) closing the valve of the plurality of first valves in fluid communication with the water source in response to reaching either a predetermined water flow time or a predetermined water flow volume as determined by the measuring device; (e) conducting a purge cycle using the control module of the reprocessing system, comprising: (i) opening the valve of the plurality of first valves in fluid communication with the pressurized air source, (ii) activating the pump to deliver pressurized air to the one or more channels via the measuring device and the one or more ports, and (iii) closing the valve of the plurality of first valves in fluid communication with the pressurized air source in response to reaching either a predetermined pressurized air flow time or a predetermined pressurized air flow volume as determined by the measuring device; (f) conducting a disinfection cycle using the control module of the reprocessing system, comprising: (i) opening the valve of the plurality of first valves in fluid communication with the disinfectant source, (ii) activating the pump to deliver disinfectant to the one or more channels via the measuring device and the one or more ports, and (iii) closing the valve of the plurality of first valves in fluid communication with the disinfectant source in response to reaching either a predetermined disinfectant flow time or a predetermined disinfectant flow volume as determined by the measuring device; and (g) repeating the rinse cycle of (d) and the purge cycle of (e).

### Example 19

The method of Example 18, wherein the control module is operable to repeat any of steps (c), (d), (e), (f), and (g) for a predetermined number of cycles.

### Example 20

The method of any one or more of Example 18 through Example 19, wherein the reprocessing system further comprises one or more second valves in fluid communication with the manifold and in fluid communication with the one or more channels of the medical device, wherein the one or more second valves are independently operable to selectively and independently direct fluid flow to the one or more channels of the medical device.

V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings. It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

1. An apparatus for reprocessing an internal channel of a medical device, the apparatus comprising:
(a) a port configured to couple with the internal channel of the medical device to provide access to the internal channel of the medical device;
(b) a measuring device coupled with the port, wherein the measuring device is configured to determine a flow volume;
(c) a pump coupled with the measuring device, wherein the pump is in fluid communication with a plurality of fluid sources, each of the fluid sources in fluid communication with a respective fluid, wherein the pump is configured to deliver the respective fluid from the fluid source to the port through the measuring device;
(d) a first plurality of valves, wherein each valve is in fluid communication with the pump and also in fluid communication with one of the plurality of fluid sources; and
(e) a control module operable to
(i) open the first plurality of valves,
(ii) close the first plurality of valves,
(iii) activate the pump to deliver the respective fluid from each of the fluid sources through a select one of the first plurality of valves to the pump and through the measuring device to the port, and
(vi) deactivate the pump to terminate delivery of the respective fluid from each of the fluid sources to the port.

2. The apparatus of claim 1, wherein the measuring device comprises a flow meter.

3. The apparatus of claim 1, wherein the measuring device is operable to determine a flow time.

4. The apparatus of claim 1, wherein the plurality of fluid sources comprises a detergent source in fluid communication with a detergent, a water source in fluid communication with a water, a pressurized air source in fluid communication with a pressurized air, and a disinfectant source in fluid communication with a disinfectant.

5. The apparatus of claim 4, wherein the control module is operable to sequentially deliver to the port: (a) the detergent from the detergent source, (b) the water from the water source, and (c) the pressurized air from the pressurized air source.

6. The apparatus of claim 5, wherein the control module is operable to sequentially deliver to the port: (d) the disinfectant from the disinfectant source, (e) the water from the water source, and (f) the pressurized air from the pressurized air source.

7. The apparatus of claim 6, wherein the plurality of fluid sources further comprises a buffer solution source in fluid communication with a buffer solution, wherein the control module is operable to deliver to the port the buffer solution from the buffer solution source.

8. The apparatus of claim 6, wherein the plurality of fluid sources further comprises an alcohol source in fluid communication with an alcohol, wherein the control module is operable to deliver to the port the alcohol from the alcohol source.

9. The apparatus of claim 7, wherein the plurality of fluid sources further comprises an alcohol source in fluid communication with an alcohol, wherein the control module is operable to deliver to the port the alcohol from the alcohol source.

10. The apparatus of claim 6, wherein the control module is operable for a predetermined number of cycles.

11. The apparatus of claim 6, wherein the control module is operable to repeat sequential delivery of the disinfectant and the pressurized air to the port for a predetermined number of cycles.

12. The apparatus of claim 1, wherein the control module is operable to rinse the pump and the measuring device with the water and then purge the pump and the measuring device of the water using the pressurized air between each delivery to the port of the detergent and the disinfectant.

13. The apparatus of claim 1, further comprising a manifold that couples the measuring device with the port.

14. The apparatus of claim 13, wherein the medical device comprises multiple internal channels, and wherein the apparatus comprises multiple ports that each couple with a respective one of the multiple internal channels of the medical device, wherein the measuring device is coupled with the multiple ports via the manifold.

15. The apparatus of claim 14, further comprising a second plurality of valves, wherein each valve of the second plurality of valves is in fluid communication with the manifold and also in fluid communication with a respective one of the multiple internal channels of the medical device, wherein each valve of the second plurality of valves is independently operable to selectively control fluid flow between the manifold and the respective one of the multiple channels of the medical device.

16. An apparatus for reprocessing an internal channel of a medical device, the apparatus comprising:
(a) a port configured to couple with the internal channel of the medical device and configured to direct a fluid to the internal channel of the medical device, wherein the fluid is a detergent, a water, a pressurized air, or a disinfectant;
(b) a measuring device coupled with the port, wherein the measuring device is configured to determine at least a select one of a flow volume and a flow time;
(c) a pump coupled with the measuring device, wherein the pump is in fluid communication with a detergent source, a water source, a pressurized air source, and a disinfectant source, wherein the pump is configured to deliver the detergent from the detergent source to the port through the measuring device, wherein the pump is further configured to deliver the water from the water source to the port through the measuring device, wherein the pump is further configured to deliver the pressurized air from the pressurized air source to the port through the measuring device, and wherein the pump is further configured to deliver the disinfectant from the disinfectant source to the port through the measuring device;
(d) a plurality of valves, wherein each valve is in fluid communication with the pump and also in fluid communication with one of the detergent source, the water source, the pressurized air source, and the disinfectant source; and
(e) a control module operable to control the plurality of valves to open and close the plurality of valves, wherein the control module is operable to:
(i) activate the pump to deliver the detergent from the detergent source through a select one of the plurality of valves to the pump and through the measuring device to the port,
(ii) terminate delivery of the detergent in response to a select one of a predetermined time limit for detergent fluid flow and a flow volume of the detergent as determined by the measuring device,
(iii) activate the pump to deliver the water from the water source through a select one of the plurality of valves to the pump and through the measuring device to the port,
(iv) terminate delivery of the water in response to a select one of a predetermined time limit for water fluid flow and a flow volume of the water as determined by the measuring device,
(v) activate the pump to deliver the pressurized air from the pressurized air source through a select one of the plurality of valves to the pump and through the measuring device to the port,
(vi) terminate delivery of the pressurized air in response to a select one of a predetermined time limit for pressurized air fluid flow and a flow volume of the pressurized air as determined by the measuring device,
(vii) activate the pump to deliver the disinfectant from the disinfectant source through a select one of the plurality of valves to the pump and through the measuring device to the port, and
(viii) terminate delivery of the disinfectant in response to a select one of a predetermined time limit for disinfectant fluid flow and a flow volume of the disinfectant as determined by the measuring device.

17. The apparatus of claim 16, wherein the control module is operable to repeat the delivery of the disinfectant and the pressurized air and from the pump to the port for a predetermined number of cycles.

18. A method of reprocessing a medical device having one or more channels, the method comprising:
(a) loading the device having the one or more channels into a reprocessing system comprising: (i) one or more ports, (ii) a manifold coupled with the one or more ports, (iii) a measuring device coupled with the manifold, (iv) a pump coupled with the measuring device, (v) a detergent source in fluid communication with a detergent, (vi) a water source in fluid communication with a water, (vii) a pressurized air source in fluid communication with a pressurized air, (viii) a disinfectant source in fluid communication with a disinfectant, (vi) a plurality of first valves each in fluid communication with the pump and a respective one of the detergent source, the water source, the pressurized air source, and the disinfectant source, and (vii) a control module operable to control the pump and the plurality of first valves;
(b) connecting each of the one or more channels of the device with the one or more ports;
(c) conducting a wash cycle using the control module of the reprocessing system, comprising: (i) opening the valve of the plurality of first valves in fluid communication with the detergent source, (ii) activating the pump to deliver detergent to the one or more channels via the measuring device and the one or more ports, and (iii) closing the valve of the plurality of first valves in fluid communication with the detergent source in response to reaching either a predetermined detergent flow volume or a predetermined detergent flow time as determined by the measuring device;
(d) conducting a rinse cycle using the control module of the reprocessing system, comprising: (i) opening the valve of the plurality of first valves in fluid communication with the water source, (ii) activating the pump to deliver water to the one or more channels via the measuring device and the one or more ports, and (iii) closing the valve of the plurality of first valves in fluid communication with the water source in response to reaching either a predetermined water flow time or a predetermined water flow volume as determined by the measuring device;
(e) conducting a purge cycle using the control module of the reprocessing system, comprising: (i) opening the valve of the plurality of first valves in fluid communication with the pressurized air source, (ii) activating the pump to deliver pressurized air to the one or more channels via the measuring device and the one or more ports, and (iii) closing the valve of the plurality of first valves in fluid communication with the pressurized air source in response to reaching either a predetermined pressurized air flow time or a predetermined pressurized air flow volume as determined by the measuring device;
(f) conducting a disinfection cycle using the control module of the reprocessing system, comprising: (i) opening the valve of the plurality of first valves in fluid communication with the disinfectant source, (ii) activating the pump to deliver disinfectant to the one or more channels via the measuring device and the one or more ports, and (iii) closing the valve of the plurality of first valves in fluid communication with the disinfectant source in response to reaching either a predetermined disinfectant flow time or a predetermined disinfectant flow volume as determined by the measuring device; and
(g) repeating the rinse cycle of (d) and the purge cycle of (e).

19. The method of claim 18, wherein the control module is operable to repeat any of steps (c), (d), (e), (f), and (g) for a predetermined number of cycles.

20. The method of claim 18, wherein the reprocessing system further comprises one or more second valves in fluid communication with the manifold and in fluid communication with the one or more channels of the medical device, wherein the one or more second valves are independently operable to selectively and independently direct fluid flow to the one or more channels of the medical device.

21-22. (canceled)
